(19) United States
(12) Patent Application Publication
(10) Pub. No.: US 2022/0211611 A1
(43) Pub. Date: Jul. 7, 2022

(54) Microcapsule With A Porous Or Hollow Core And A Shell Containing A Component Releasing Gas Upon Contact With An Acid

(71) Applicant: 3M INNOVATIVE PROPERTIES COMPANY, St. Paul, MN (US)

(72) Inventors: Kai U. Claussen, Düsseldorf (DE); Silke D. Mechernich, Düsseldorf (DE); Hassan Sahouani, Hastings, MN (US)

(21) Appl. No.: 17/595,217

(22) PCT Filed: May 27, 2020
(86) PCT No.: PCT/IB2020/055044

§ 371 Date: Nov. 11, 2021

(30) Foreign Application Priority Data
Jun. 4, 2019 (EP) 19178074.1

(51) Int. Cl. A61K 9/00; A61K 9/48; A61K 9/50

(52) U.S. Cl. CPC A61K 9/0004; A61K 9/5031; A61K 9/4866

(57) ABSTRACT

The invention relates to a microcapsule comprising a hollow or porous core, the hollow or porous core being composed of a crosslinked polymeric material and containing a component to be released, a shell, the shell being composed of a polymeric material containing a component, which is able to produce gas upon exposure to acid. The microcapsule can be used for formulating dental materials.

### FIELD OF THE INVENTION

The invention generally relates to a pH-sensitive microcapsule, a paste/paste system containing such a microcapsule and its use for producing a curable composition comprising e.g. a redox-initiator system.

The microcapsule has a porous or hollow core which is covered by a polymeric shell which contains a component being able to produce or release gas upon contact with an acid.

### BACKGROUND

The use of microcapsules for storing components such as components of redox initiator systems is generally known.

U.S. Pat. No. 5,154,762 (Mitra et al.) describes in example 11 the microencapsulation of ascorbic acid in cellulose acetate butyrate. It is stated that although the use of water-insoluble encapsulants may initially seem inappropriate in water-based cements, it was found that vigorous mechanical mixing generally is sufficient to break apart the capsule walls and permit adequate release of the encapsulated reducing agent or oxidizing agent and subsequent cure of the cement. This technology is particularly useful for preparing powder compositions. However, compared to paste/paste systems powder compositions are easier to prepare and stabilize due to the physical separation of the powder components to be mixed. In contrast thereto, pasty compositions are typically manufactured by kneading processes where high shear forces are applied onto the microcapsules.

The microcapsules described in the prior art, in particular those suggested for storing components of a redox-initiator system, are typically not sufficiently stable to survive such high shear forces.

On the other hand, when mixing paste/paste compositions the applied mixing forces are often not sufficiently strong enough for breaking the microcapsules to enable the release of the active reagents.

Therefore, the technology used for producing powder compositions can typically not be used for paste/paste compositions.

Other references which describe the use and production of microcapsules are:

U.S. Pat. No. 9,422,411 B2 (Sahouani et al.) relates to porous polymeric particles that can be hydrophilic or hydrophobic. The porous polymeric particles can be used for the storage and delivery of various active agents or for moisture management. Reaction mixtures for forming the porous polymeric particles, methods of making the porous polymeric particles, and articles containing the porous polymeric particles are also provided.

US 2016/088836 A1 (Sahouani et al) describes polymeric composite particles that can be used for the storage and delivery of various biologically active agents. The polymeric composite particles contain a porous polymeric core and a coating layer around the porous polymeric core. The porous polymeric composite particles typically further include a biologically active agent positioned within the porous polymeric core but not covalently bonded to the porous polymeric core. The biologically active agent can be released from the polymeric composite particle by diffusing out of the porous polymeric core through the coating layer.

WO 2016/053830 A1 (3M IPC) describes articles that include a fibrous substrate and porous polymeric particles. At least 50% of the porous polymeric particles are bound to the fibrous substrate. Methods of making the articles are provided that include providing porous polymeric particles, providing a fibrous substrate, and binding the porous polymeric particles to the fibrous substrate. The articles can be used for fluid management.

U.S. Pat. No. 6,391,288 B1 (Miyazawa et al) describes the preparation of microcapsules comprising an inner oil phase, a water phase and an outer oil phase. Ascorbic acid (in the water phase) is encapsulated. The microcapsules have a fracture strength of 10-500 g/cm² or 500-2,000 g/cm² or 2,000-5,000 g/cm². 0/w emulsions are used to produce the microcapsules. The release of the active ingredient such as ascorbic acid can be triggered by an appropriate rupture strength.

CN 108276965 A describes a process for preparing a microcapsule, wherein in a first step a porous expanded perlite is immersed in liquid n-dodecly alcohol, subjected to suction filtration, rapidly cooled and fixed in liquid nitrogen. In a second step a coating liquid is formed by mixing an urea-formaldehyde resin with vegetable oil, nano calcium carbonate and a coupling agent. In a third step, the coating liquid together with a curing agent solution is sprayed onto the capsule core to form a core-shell structure.

### SUMMARY OF INVENTION

There is a desire for microcapsules which can be used for producing storage-stable pasty compositions.

There is also a desire for a microcapsule which is sufficiently mechanically stable and allows a fast release on demand of a reagent or component stored in the microcapsule. Further, the release on demand of the component to be released should be sufficiently fast. This object is addressed by the microcapsules and related processes described in the claims and the present text.

In one embodiment the invention features a microcapsule as described in the present text and claims, the microcapsule comprising a hollow or porous core, the hollow or porous core being composed of a polymeric material and comprising a component to be released, a shell, the shell being composed of a polymeric material and comprising a component, which produces gas upon exposure to acid.

A further embodiment of the invention is directed to a process of producing the microcapsule as described in the present text and claims, the process comprising the steps of: providing a particle with a hollow or porous core and a component of a redox-initiator system, having the particle absorb the component of the redox-initiator system, coating the particle containing the absorbed component of the redox-initiator system with a polymeric coating material comprising a component, which release gas upon exposure to acid.

In another embodiment, the invention relates to a kit of parts as described in the present text and claims, the kit of parts comprising a Catalyst Paste and a Base Paste, the Catalyst Paste comprising the microcapsule as described in the present text and claims comprising a first component of a redox-initiator system, the Base Paste comprising acidic component and a second component of the redox-initiator system.

The invention is also related to a process of curing a curable composition as described in the present text and claims, the process comprising the steps: providing a Catalyst Paste comprising the microcapsules as described in the present text and claims and a Base Paste comprising an acidic component, wherein either the Catalyst Paste or the Base Paste or the Catalyst Paste and the Base Paste comprise curable components, mixing the Catalyst Paste and the Base Paste.

The invention is also directed to the use of the microcapsules as described in the present text and claims for producing a curable composition comprising a redox-initiator system, in particular a dental or orthodontic composition, such as a dental or orthodontic cement, adhesive or filling material.

The microcapsules essentially consist of a mechanically stable hollow or porous core which may be filled with a component to be released and covered with a polymeric shell that typically swells or dissolves in an aqueous composition (e.g. a composition having a water content of 10 wt. % or more than 10 wt. %).

To improve the release of the component to be released, the shell further contains a component which releases gas upon exposure to an acidic environment. Such an accelerated release is sometimes called burst-release.

Unless defined differently, for this description the following terms shall have the given meaning:

A “hollow core” refers to polymeric particles having a polymeric outer shell surrounding an inner region or cavity that is not polymeric.

A “porous core” refers to polymeric particles having a polymeric structure containing voids or pores.

As used herein, “(meth)acryl” is a shorthand term referring to “acryl” and/or “methacryl”. For example, a “(meth) acryloxy” group is a shorthand term referring to either an acryloxy group (i. e., CH₂═CH—C(O)—O—) and/or a methacryloxy group (i. e., CH₂═C(CH₃)—C(O)—O—).

An “initiator” is a substance being able to initiate a chemical reaction, preferably via a free radical reaction. The initiator can be a single compound or can comprise more than one component, such as a combination of a sensitizing agent with a reducing agent. Depending on the reaction conditions chosen (e.g. pH-value >7 or pH-value <7) different initiators can be preferred.

A “redox initiator system” is defined as the combination of reducing agent(s) and oxidizing agent(s) being located on the application part of the application device. If present, transition metal component(s) are also regarded as components of the redox initiator system.

As used herein, “hardening” or “curing” a composition are used interchangeably and refer to polymerization and/or crosslinking reactions including, for example, photopolymerization reactions and chemical polymerization techniques (e. g., ionic reactions or chemical reactions forming radicals, effective to polymerize ethylenically unsaturated compounds) involving one or more materials included in the composition.

A “dental composition” or a “composition for dental use” or a “composition to be used in the dental field” is any composition which can be used in the dental field. In this respect, the composition should be not detrimental to the patients' health and thus be free of hazardous and toxic components being able to migrate out of the composition.

Examples of dental compositions include permanent and temporary crown and bridge materials, artificial crowns, anterior or posterior filling materials, adhesives, mill blanks, lab materials, luting agents and orthodontic devices. Dental compositions are typically hardenable compositions, which can be hardened at ambient conditions, including a temperature range of 15 to 50° C. or from 20 to 40° C. within a time frame of 30 min or 20 min or 10 min. Higher temperatures are not recommended as they might cause pain to the patient and may be detrimental to the patient's health. Dental compositions are typically provided to the practitioner in comparable small volumes, that is volumes in the range of 0.1 to 100 ml or 0.5 to 50 ml or 1 to 30 ml. Thus, the storage volume of useful packaging devices is within these ranges.

The term “compound” or “component” is a chemical substance which has a particular molecular identity or is made of a mixture of such substances, e.g., polymeric substances.

A “polymerizable component” is any component which can be cured or solidified e.g. by heating to cause polymerization or chemical crosslinking, or e.g. by radiation-induced polymerization or crosslinking, or e.g. using a redox initiator or by any other radical forming process. A radically polymerizable component may contain only one, two, three or more radically polymerizable groups. Typical examples of radically polymerizable groups include unsaturated carbon groups, such as a vinyl group being present e.g. in a (methyl)acrylate group.

A “monomer” is any chemical substance which can be characterized by a chemical formula, bearing radically polymerizable unsaturated groups (including (meth)acrylate groups) which can be polymerized to oligomers or polymers thereby increasing the molecular weight. The molecular weight of monomers can usually simply be calculated based on the chemical formula given.

“Polymer” or “polymeric material” are used interchangeably to refer to a homopolymer, copolymer, terpolymer etc.

A “derivative” or “structural analogue” is a chemical compound showing a chemical structure closely related to the corresponding reference compound and containing all featured structural elements of the corresponding reference compound but having small modifications like bearing additional chemical groups like e.g. alkyl moieties, Br, Cl, or F or not bearing chemical groups like e.g. alkyl moieties in comparison to the corresponding reference compound. That is, a derivative is a structural analogue of the reference compound. A derivative of a chemical compound is a compound comprising the chemical structure of said chemical compound.

A component comprising an “ascorbic acid moiety” is a component comprising the following structural element:

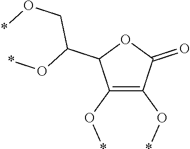 [SMILES: *OCC(O*)C1OC(=O)C(O*)=C1O*]

wherein the symbol “*” indicates a connection to another chemical moiety or atom.

“Ambient conditions” mean the conditions which the inventive composition is usually subjected to during storage and handling. Ambient conditions may, for example, be a pressure of 900 to 1100 mbar, a temperature of −10 to 60° C. and a relative humidity of 10 to 100%. In the laboratory, ambient conditions are adjusted to 23° C. and 1013 mbar. In the dental and orthodontic field ambient conditions are reasonably understood as a pressure of 950 to 1050 mbar, temperature of 15 to 40° C. and relative humidity of 20 to 80%.

As used herein, “a”, “an”, “the”, “at least one” and “one or more” are used interchangeably. The terms “comprise” or “contain” and variations thereof do not have a limiting meaning where these terms appear in the description and claims. The term “comprising” also includes the more limited expressions “consisting essentially of” and “consisting of”.

Also herein, the recitations of numerical ranges by endpoints include all numbers subsumed within that range (e.g., 1 to 5 includes 1, 1.5, 2, 2.75, 3, 3.80, 4, 5, etc.). The terms “comprise” or “contain” do not have a limiting meaning where these terms appear in the description and claims. That is, further components can be present. The term “comprise” shall include also the terms “consist essentially of” and “consist of” “Consisting essentially of” means that specific further components can be present, namely those which do not materially affect the essential characteristic of the article or composition. “Consisting of” means that no further components should be present.

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and used in the specification and claims are to be understood as number as such and also as being modified by the term “about.”

“And/or” means one or both. E.g., the expression component A and/or component B refers to a component A alone, component B alone, or to both component A and component B.

### BRIEF DESCRIPTION OF FIGURES

FIG. 1 shows a SEM picture of microcapsules described in the present text containing a component of a redox-initiator system and being coated with a water-soluble polymeric material comprising particles which produce gas upon exposure to acid.

FIG. 2 shows a SEM picture of microcapsules described in the present text containing a component of a redox-initiator system and being coated with another water-soluble polymeric material comprising particles which produce gas upon exposure to acid.

### DETAILED DESCRIPTION

The microcapsules and its use described in the present text are advantageous for a couple of reasons.

The microcapsules contain a porous or hollow core which is suitable to absorb or store a component to be released, e.g. an active reagent, such as a component of a redox-initiator system.

The microcapsules further contain a shell or coating which prevents the component to be released to migrate out of the porous or hollow core during storage. Thus, a reaction of the encapsulated components with component(s) surrounding the microcapsule is avoided, thereby improving shelf-life stability.

The microcapsules are sufficiently mechanically stable and survive shear forces which typically occur during production process involving a kneading step, e.g. when preparing pasty compositions.

The polymeric shell which contains a component which produces gas upon exposure to an acidic environment allows the release of the component to be released from the porous or hollow core on demand.

Due to this property, the microcapsules described in the present text can also be regarded as pH-sensitive microcapsules.

This can be advantageous for e.g. redox-initiator systems contained in a dental two-part paste/paste composition that contain an acidic component.

Thus, the microcapsules described in the present text can help to overcome challenges associated e.g. with the production of redox-curable paste/paste compositions.

Further, due to the presence of a component in the polymeric shell, a component which releases gas if brought in contact with an acid, the component to be released can be released more rapidly from the microcapsule if the microcapsule is brought in contact with an acidic environment. The gas produced by the gas-producing component helps to widen, perforate and/or to remove the polymeric shell from the porous or hollow core.

For producing storage-stable paste/paste compositions which contain a redox-initiator system a fast release of the redox-initiator components is often desired to ensure the hardening of the curable composition within an appropriate time.

The core-shell microcapsule described in the present text comprises a porous or hollow core.

The porous or hollow core is composed of a polymeric material, i.e. a crosslinked matrix. It was found that a crosslinked matrix has sufficient mechanical stability to withstand shear forces which typically occur during mixing or kneading processes.

The microcapsules can typically be characterized by the following features alone or in combination:

a) shape: essentially spherical;

b) diameter: 1 to 200 μm or 1 to 100 μm or 5 to 100 μm or 5 to 50 μm or 5 to 25 μm;

c) pore size of porous core material: 10 to 200 nm or 20 to 200 nm or 50 to 200 nm;

d) mechanically stable.

A combination of the features a) and b) or a) and c) or b) and c) or a), b) and c) or a), b), c) and d) is sometimes preferred.

According to one embodiment, the microcapsules is characterized by the following features:

a) shape: essentially spherical;

b) diameter: 10 to 100 μm;

c) pore size of porous core material: 10 to 200 nm;

d) mechanically stable.

The shape, diameter and pore size can be evaluated by microscopy, in particular by scanning electron microscopy (SEM). If desired, the diameter and particle size distribution can be determined by light scattering.

A microcapsule is mechanically stable, if it is able to survive high shear forces, which typically occur during preparing pastes in a mixing or kneading machine. A suitable test is described in the example section.

Mechanical stability can be obtained, e.g. if a crosslinked polymeric material, in particular a highly crosslinked polymeric material is used.

The polymeric material of the porous or hollow core is typically a (meth)acrylate, that is, the polymerization product of polymerizable monomers containing (meth)acrylate moieties.

Suitable (meth)acrylates, which may be present in the polymeric material include

CH₂═C(R¹)—(CO)—O[—CH₂—CH₂—O]ₚ—(CO)—C(R¹)═CH₂ (1)

wherein p is an integer equal to at least 1; R¹ is hydrogen or alkyl;

H₂═CR¹—(CO)—O—Y—R² (2)

wherein R¹ is hydrogen or methyl, Y is a single bond, alkylene, oxyalkylene, or poly(oxyalkylene) and R² is a carbocyclic group or heterocyclic group;
and mixtures thereof.

The term “alkylene” refers to a divalent group that is a radical of an alkane and includes groups that are linear, branched, cyclic, bicyclic or combinations thereof. Suitable alkylene groups are selected from C₁ to C₂₀ or C₁ to C₁₆ or C₁ to C₁₂ or C₁ to C₁₀ or C₁ to C₈ or C₁ to C₆ or C₁ to C₄ moieties.

The material of the porous or hollow core is typically obtained by emulsion polymerization of suitable monomers. Suitable monomers include those described above.

According to one embodiment, the polymeric material of the porous or hollow core is obtained by polymerizing the components according to formulas (1) and (2) optionally in the presence of other components.

Other components include non-ionic surfactants and/or components of formula (3)

HO[—CH₂—CH(OH)—CH₂—O]ₙ—H (3)

wherein n is an integer equal to at least 1.

The polymerization is typically initiated by an initiator for free radical polymerization.

According to a further embodiment, the polymeric material of the porous or hollow core is the polymerized product of a reaction mixture comprising:
a) a first phase comprising
   i) a compound of formula (3); and
   ii) a non-ionic surfactant; and
b) a second phase dispersed in the first phase, wherein the second phase comprises
   iii) a monomer composition comprising a monomer of formula (1);
   iv) a polypropylene glycol, preferably having a Mw of at least 500 g/mol; and
i) optionally a further monomer of formula (2).

Suitable processes for producing microcapsules having a porous or hollow core are e.g. described in U.S. Pat. No. 9,422,411 B2 (Sahouani et al.) or US 2016/008836 A1 (Sahouani et al.). The content of these references is herewith incorporated by reference. The porous microcapsules are typically produced via an emulsion polymerization process.

The core-shell microcapsule described in the present text also comprises a shell. The shell covers the porous or hollow core of the microcapsules.

The shell can typically be characterized by the following features alone or in combination:
a) thickness: 0.1 to 5 μm or 0.5 to 4 μm or 1 to 3 μm;
b) coverage: covering at least 85% or at least 90 or at least 95% or at least 99% of the surface of the porous or hollow core;
c) insoluble in organic monomers (e.g. those described further down below as polymerizable components without acidic moiety; e.g. TEGDMA);
d) soluble or swellable in an aqueous composition, e.g. a composition with a water content of 10 wt. % or more.

A shell characterized by the combination of the following features is sometimes preferred: a) and b); a) and c); b) and c); a), c) and d).

Using a shell material being soluble in an acidic aqueous composition can sometimes be preferred.

According to one embodiment, the microcapsule shell is characterized by the following features:
a) thickness: 0.1 to 5 μm;
b) insoluble in TEGDMA.

If desired, the thickness of the shell can be determined by Scanning Electron Microscopy (SEM), Transmission Electron Microscopy (TEM) or Secondary Ion Mass Spectroscopy (SIMS).

If desired, the extent of coverage of the shell can be determined by Scanning Electron Microscopy (SEM).

The shell is composed of a polymeric material. The shell protects the hollow or porous core of the microcapsule. According to one embodiment, the polymeric material is an acid-sensitive material.

An acid-sensitive polymeric material is a polymeric material which dissolves, swells or weakens if brought in contact with an acidic component.

Acid-sensitive polymeric materials are typically characterized by the following features alone or in combination:
a) dissolving in a composition having a pH in the range of 1 to 6 or of 1 to 4;
b) being stable in composition having a pH in the range of 14 to 7;
c) having a molecular weight Mw of 10,000 to 1,000,000 g/mol or 20,000 to 500,000 g/mol or 50,000 g/mol to 300,000 g/mol;
d) glass temperature (Tg) below 200° C. or below 180° C. or below 150° C. or below 100° C.

An acid-sensitive polymeric material characterized by the combination of the following features is sometimes preferred: a) and b); a) and c); b) and c); a), b) and c).

The molecular weight Mw is typically provided by the supplier of these materials or can be determined, if desired, by gel permeation chromatography (GPC) using e.g. a polystyrene standard.

According to one embodiment, the acid-sensitive polymeric material of the shell is characterized by the following features:
a) dissolving in a composition having a pH in the range of 1 to 4;
b) having a molecular weight Mw of 10,000 to 1,000,000 g/mol;
c) glass temperature T_{g} below 150° C.

Examples of acid-sensitive materials include co-polymer(s) of methyl (meth)acrylate and diethylaminoethyl (meth)acrylate (e.g. those sold under the trade name Kollicoat™ Smartseal), co-polymer(s) of methyl (meth)acrylate, butyl methacrylate and dimethylaminoethyl (meth)acrylate (e.g. those sold under the trade name Eudragit™ E), poly(2-(dimethylamino)ethyl methacrylate), cellulose acetate phthalate, sodium carboxymethyl cellulose, hydroxy propyl methyl cellulose phthalate, poly(vinyl acetate phthalate), poly(4-vinyl pyridine), chitosan, and mixtures thereof.

If desired, the pH-sensitive polymeric shell can be removed or broken up by bringing the shell in contact with an acidic component. According to another embodiment, the polymeric material is a basic-sensitive material.

Basic-sensitive materials are typically characterized by the following features alone or in combination:
a) dissolving in a composition having a pH in the range of 14 to <7 or of 12 to 8;
b) being stable in composition having a pH in the range of 1 to 7 or in the range of 1 to 5;
c) being polymeric, e.g. having a molecular weight Mw of 10,000 to 1,000,000 g/mol or 20,000 to 500,000 g/mol or 50,000 g/mol to 300,000 g/mol;
d) glass temperature T_{g}: below 200° C. or below 180° C. or below 150° C. or below 100° C.

A basic-sensitive polymeric material characterized by the combination of the following features is sometimes preferred: a) and b); a) and c); b) and c); a), b) and c).

According to one embodiment, the basic-sensitive polymeric material is characterized by the following features:
a) dissolving in a composition having a pH in the range of 12 to 8;
b) being polymeric, e.g. having a molecular weight Mw of 10,000 to 1,000,000 g/mol;
c) glass temperature T_{g} below 150° C.

Examples of basic-sensitive materials include co-polymer(s) of methacrylic acid and methyl (meth)acrylate (e.g. those sold under the trade name Eudragit™ L, S), co-polymer(s) of methacrylic acid and alkyl (e.g. C₁₋₆) (meth)acrylate, co-polymer(s) of methacrylic acid, methyl (meth)acrylate and methyl acrylate (e.g. those sold under the trade name Eudragit™ FS 30D), poly(acrylic acid), poly(sulfonic acid), poly(styrene sulfonic acid, poly(2-hydroxylethyl methacrylate) phosphate, hyaluronic acid, and mixtures thereof.

If desired, the pH-sensitive polymeric shell can be removed or broken up by bringing the shell in contact with a basic component.

According to one embodiment, the polymeric material is soluble in an aqueous composition (e.g. a composition with a water content of 10 wt. % or more). Such a polymeric material is regarded as water-soluble.

Water-soluble materials are typically characterized by the following features alone or in combination:
a) being polymeric, e.g. having a molecular weight Mw of 10,000 to 1,000,000 g/mol or 20,000 to 500,000 g/mol or 50,000 g/mol to 300,000 g/mol;
b) glass temperature T_{g} below 200° C. or below 180° C. or below 150° C. or below 100° C.

Examples of water-soluble materials include hydroxypropyl modified pea-starch, graft co-polymer(s) of vinyl alcohol and ethylene glycol and mixtures thereof (e.g. those sold under the trade name Kollicoat™ IR or Lycoat™ RS 780).

The use of an acid-sensitive polymeric material for the shell of the microcapsule is sometimes preferred, in particular if the formulation of a paste/paste composition is intended, where one of the pastes contains an acidic component.

The acidic component contained in that paste can then be used as a further trigger to remove or weaken the shell upon mixing the pastes, which results in the release of the encapsulated component to be released. The shell comprises a component, which produces gas upon exposure to acid.

The presence of such a component in the shell helps to improve the release of the component to be release and stored in the hollow or porous core of the microcapsule.

The release of this component can be even further improved, if the pH-sensitive polymeric material of the shell is acid-sensitive.

If such a microcapsule is brought in contact with an acidic environment, not only the pH-sensitive polymeric material is broken up but the break-up is accelerated by the component which produces gas upon exposure to the acid.

The component which produce gas upon exposure to an acid can typically be characterized by the following features alone or in combination:
a) having a molecular weight in the range of 68 to 200 g/mol;
b) having the shape or particles with a particle size in the range of 5 nm to 20 μm or 5 nm to 25 μm or 5 μm to 10 μm or 5 nm to 5 μm or 5 nm to 3 μm or 5 nm to 1 μm.

It was found that using a component with a molecular weight in the above range typically react sufficiently fast with acid to produce gas.

It was also found that by using components having a small particle size the reaction with acid to produce gas can be improved.

The component which produce gas upon exposure to an acid typically comprising a moiety selected from carbonate, hydrogen carbonate. The gas which is produced is typically CO₂.

The component which produce gas upon exposure to an acid can be an organic or inorganic component, wherein inorganic components are sometimes preferred.

Examples of components which produce gas upon exposure to an acid include alkali metal (e.g. Li, Na, K) and earth alkali metal (e.g. Mg, Ca) carbonates as well as Zn carbonate and hydrogen carbonates such as Li₂CO₃, Na₂CO₃, K₂CO₃, MgCO₃, CaCO₃, ZnCO₃, LiHCO₃, NaHCO₃, KHCO₃ and mixtures thereof.

If the microcapsules are to be used in the medical or dental field, the components should be sufficiently biocompatible and essentially non-toxic in the amount used.

The use of the following components is sometimes preferred as they have been proven to be very effective for the desired use: CaCO₃, Na₂CO₃, NaHCO₃.

The ratio of polymeric material to component which produce gas upon exposure to an acid is typically in a range of 2:1 to 20:1 or 4:1 to 10:1 with respect to weight.

Such a ratio is considered to provide a good balance between the protection function of the shell and the ability to rapidly release the component to be released upon exposure to an acid.

The microcapsule contains a component to be released. The component to be released is contained in the porous or hollow core of the microcapsule.

Any kind of component to be released can be stored in the microcapsule which does not negatively interact with the material of the core or shell of the microcapsule.

The release of this component can be accomplished by bringing the polymeric shell containing gas releasing compounds in contact with either an acidic or basic component.

According to one embodiment the porous or hollow core contains one component of a redox-initiator system. Such a component is sometimes referred to as active agent.

A redox-initiator system typically comprises oxidizing agent(s) and reducing agent (s) and sometimes transition metal(s). According to one embodiment, the porous or hollow core contains oxidizing agent(s).

The nature and structure of the oxidizing agent(s) is not particularly limited unless the desired result cannot be achieved.

Suitable oxidizing agents include organic and inorganic peroxides, persulfate component(s) and mixtures thereof.

Generally, all peroxide(s), i.e. inorganic and organic peroxides, which can be incorporated or absorbed by the microcapsules can be used.

In contrast to inorganic peroxides, organic peroxide(s) do not comprise metals or metal ions. Thus, organic peroxides typically only comprise C, O, H and optionally halogens (e.g. F, Cl, Br).

Organic peroxides which can be used include hydroperoxide(s), ketone peroxide(s), diacyl peroxide(s), dialkyl peroxide(s), peroxyketal(s), peroxyester(s) and peroxydicarbonate(s).

Di-peroxides, which can be used include di-peroxides comprising the moiety R₁—O—O—R₂—O—O—R₃, with R₁ and R₃ being independently selected from H, alkyl (e.g. C₁ to C₆), branched alkyl (e.g. C₁ to C₆), cycloalkyl (e.g. C₅ to C₁₀), alkylaryl (e.g. C₇ to C₁₂) or aryl (e.g. C₆ to C₁₀) and R₂ being selected from alkyl (e.g. (C₁ to C₆) or branched alkyl (e.g. C₁ to C₆).

Examples of ketone peroxides include methyl ethyl ketone peroxide, methyl isobutyl ketone peroxide, methyl cyclohexanone peroxide, and cyclohexanone peroxide.

Examples of peroxyesters include □-cumylperoxyneodecanoate, t-butyl peroxypivarate, t-butyl peroxyneodecanoate, 2,2,4-trimethylpentylperoxy-2-ethyl hexanoate, t-amylperoxy-2-ethyl hexanoate, t-butylperoxy-2-ethyl hexanoate, di-t-butylperoxy isophthalate, di-t-butylperoxy hexahydroterephthalate, t-butylperoxy-3,3,5-trimethylhexanoate, t-butylperoxy acetate, t-butylperoxy benzoate and t-butylperoxymaleic acid.

Examples of peroxidicarbonates include di-3-methoxy peroxidicarbonate, di-2-ethylhexyl peroxydicarbonate, bis(4-t-butylcyclohexyl)peroxidicarbonate, diisopropyl-1-peroxydicarbonate, di-n-propyl peroxidicarbonate, di-2-ethoxy ethyl-peroxidicarbonate, and di allyl peroxidicarbonate.

Examples of diacyl peroxides include acetyl peroxide, benzoyl peroxide, decanoyl peroxide, 3,3,5-trimethylhexanoyl peroxide, 2,4-dichlorobenzoyl peroxide and lauroylperoxide.

Examples of dialkyl peroxides include di-t-butyl peroxide, dicumylperoxide, t-butyl cumyl peroxide, 2,5-dim ethyl-2,5-di(t-butylperpoxy)hexane, 1,3-bis(t-butylperoxyisopropyl)benzene and 2,5-dimethyl-2,5-di(t-butylperoxy)-3-hexane.

Examples of peroxyketals include 1,1-bis(t-butylperoxy)-3,3,5-trimethylcyclohexane, 1,1-bis(t-butylperoxy)cyclohexane, 2,2-bis(t-butylperoxy)butane, 2,2-bis(t-butylperoxy)octane and 4,4-bis(t-butylperoxy)valeric acid-n-butylester.

According to one embodiment, the organic peroxide is a hydroperoxide, in particular a hydroperoxide comprising the structural moiety R—O—O—H with R being (e.g. C₁ to C₂₀) alkyl, (e.g. C₃ to C₂₀) branched alkyl, (e.g. C₆ to C₁₂) cycloalkyl, (e.g. C₇ to C₂₀) alkylaryl or (e.g. C₆ to C₁₂) aryl.

Examples of suitable organic hydroperoxides include t-butyl hydroperoxide, t-amyl hydroperoxide, p-diisopropylbenzene hydroperoxide, cumene hydroperoxide, pinane hydroperoxide, p-methane hydroperoxide and 1,1,3,3-tetramethylbutyl hydroperoxide.

Suitable peroxodisulfate components and/or peroxodiphosphate components and/or mixtures thereof, which can be used include organic and/or inorganic components.

Suitable examples include ammonium, sodium, and potassium peroxodisulfate components and/or peroxodiphosphate components. Sodium peroxodisulfate is sometimes preferred. Alternatively, the hollow or porous core contains reducing agents(s).

The nature and structure of the reducing agent(s) is not particularly limited unless the desired result cannot be achieved.

Suitable reducing agents include organic and inorganic component(s) and mixtures thereof. The reducing agent is typically a solid at ambient conditions (23° C.; 1013 hPa).

Reducing agents (s) which may be contained in the porous or hollow core include ascorbic acid component(s), tertiary amine component(s), sulfinate component(s), sulphite component(s), borane component(s), (thio)urea component(s), and (thio)barbituric acid component(s), saccharin and metal salts thereof.

Component(s) comprising an ascorbic acid moiety such as salts and esters of ascorbic acid, ethers, ketals, or acetals are sometimes preferred.

Suitable salts include the alkali metal and earth alkali metal salts like Na, K, Ca and mixtures thereof.

Esters of ascorbic acid include those, which are formed by reacting one or more of the hydroxyl functions of ascorbic acid with a carboxylic acid, in particular the C₂ to C₃₀ carboxylic acid.

Suitable examples of C₂ to C₃₀ carboxylic acids include the fatty acids, like caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

In particular preferred are these ascorbic acid moiety containing components, which can be easily dissolved in or mixed with the remaining resin matrix comprising polymerizable components.

That is, using an ascorbic acid moiety containing component having in addition a hydrophobic moiety can sometimes be preferred. Suitable hydrophobic moieties include saturated and unsaturated aliphatic residues (e.g. C₂ to C₃₀ or C₁₂ to C₃₀). Those ascorbic acid derivatives may also function as surface-active substances (substances having a so-called “head/tail structure”). Particularly preferred are sometimes ascorbyl palmitate, ascorbyl stearate, mixtures and salts thereof.

If the oxidizing agents(s) are brought in contact with the reducing agents(s), a redox-reaction typically starts. Such a redox-reaction is suitable to initiate the curing of curable components resulting in the crosslinking of the curable components.

Other components of a redox-initiator system which can be present and be contained in the porous or hollow core include transition metal components.

Suitable transition metal component(s) include organic and/or inorganic salt(s) selected from titanium, vanadium, chromium, manganese, iron, cobalt, nickel, copper and/or zinc, with copper and iron being sometimes preferred.

Useful salts include acetate(s), chloride(s), sulphate(s), benzoate(s), acetylacetonate(s), naphthenate(s), carboxylate(s), bis(1-phenylpentan-1,3-dione) complexes, salicylate(s), complexes with ethylenediamine tetra acetic acid of either of the transition metals and mixtures thereof.

According to one embodiment, the transition metal component is in an oxidation stage, which allows the component to be reduced. Useful oxidation stages include +2, +3, +4, +5, +6 and +7, as applicable.

Copper component(s) are sometimes preferred. The oxidation stage of copper in the copper component(s) is preferably +1 or +2.

Typical examples of copper component(s) which can be used include salts and complexes of copper including copper acetate, copper chloride, copper benzoate, copper acetylacetonate, copper naphthenate, copper carboxylates, copper bis(1-phenylpentan-1,3-dione) complex (copper procetonate), copper salicylate, complexes of copper with thiourea, ethylenediamine tetra acetic acid and/or mixtures thereof. The copper compounds can be used in hydrated form or free of water. Especially preferred is copper acetate.

If desired, the porous or hollow polymer particles can also be filled with other components, for example a dye(s), or crosslinker(s), fluoride releasing agent(s). Suitable dye(s) and fluoride releasing agent(s) are described in the text below. The microcapsule described in the present text can be produced as follows:

Porous or hollow particles composed of a polymeric material and component(s) to be released such as components of a redox-initiator system as described in the present text are provided.

The porous or hollow particle are treated with the component(s) to be released in a manner enabling the porous particles to absorb the component(s).

If the component(s) to be released are in a solid or high viscous state, the component(s) are typically dissolved in a solvent first.

After the treatment, the solvent is typically evaporated, e.g. by drying the treated microcapsules.

Suitable solvents include water and low-boiling solvents. Low-boiling solvents typically have a boiling point at ambient pressure below 80° C.

Suitable solvents include water, methylene chloride, low boiling ethers (e.g. tetrahydrofuran, methyl tert. butyl ether), alcohols (e.g. methanol, ethanol, iso- and n-propanol), and mixtures thereof.

After the treatment, the component(s) to be released are located in or absorbed by the pores of the porous particles.

If desired, the treating process can be characterized by the following features, alone or in combination:
a) duration: 5 to 60 min;
b) temperature: 20 to 60° C.;
c) pressure: ambient pressure;
d) stirring the mixture.

The combination of the following features is sometimes preferred: a) and b); a), b) and c); or a), b), c) and d).

The porous or hollow particles having the component(s) to be released contained in the pores is then treated with a polymeric coating agent. Examples of polymeric coating agents include those mentioned above.

To the polymeric coating agent the component which produces gas upon contact with an acid is added. If desired, a solvent (including those mentioned above) may be added, as well. Further, if desired, a surfactant can be added.

The addition of a surfactant can be beneficial in that it may facilitate a more homogeneous distribution of the component which produces gas upon contact with an acid in the coating composition, and thus facilitate the coating process. As a result, the component which produces gas upon contact with an acid may be distributed in the coating layer more evenly.

The nature of the surfactant is not particularly limited unless the desired effect cannot be achieved.

Suitable surfactants include ionic surfactants (e.g., sulfate, sulfonate, phosphate, carboxylate esters, quaternary ammonium salts), amphoteric surfactants (e.g., sultaines, betaines) and non-ionic surfactants, wherein water-soluble surfactants are preferred. In particular, non-ionic surfactants were found be suitable.

Non-ionic surfactants typically have covalently bonded oxygen-containing hydrophilic groups, which are bonded to hydrophobic parent structures. Compared to other surfactants, non-ionic surfactants often foam less strongly.

Examples of non-ionic surfactants which can be used include alkyl polyglycosides, fatty amine ethoxylates, fatty alcohol ethoxylates, fatty acid alkanolamides, castor oil ethoxylates, alcohol ethoxylates/propoxylates and blends thereof (e.g. blend of decyl and undecyl glucosides; APG™ 325, BASF).

If a surfactant is used, it is typically used in only a small amount, e.g. 2 to 10 wt. % or 5 to 8 wt. % with respect to the amount of the component which produces gas upon contact with an acid.

At this stage, the component which is able to produce gas upon contact with an acid may have a particle size in the range of 5 nm to 30 μm. Due to a partial dissolution of the particles in the coating agent and/or solvent, the size of the particles typically shrinks. Thus, the particle size of the initially provided particles can be larger than the particle size which these particles later have after incorporation in the polymeric shell.

If desired, the coating process can be characterized by the following features, alone or in combination:
a) the coating process being done by spray-drying;
b) duration: 0.1 to 10 h, or 0.2 to 5 h;
c) temperature: 20 to 90° C. or 30 to 80° C. or 40 to 70° C.;
d) pressure: ambient pressure (e.g. 900 to 1030 hPa).

The combination of the following features is sometimes preferred: b) and c); or a), b) and c); or a), b), c) and d).

The spray drying is typically done at a temperature around the T_{g} (glass temperature) of the polymer.

It has been found that this temperature is often appropriate to ensure an appropriate encapsulation and to obtain a smooth and homogenous surface.

Thus, typical coating agents are polymers, copolymers or waxes with glass transition temperatures (T_{g}) below 200° C. and a molecular weight (Mw) in the range of 20,000 to 500,000 g/mol.

The spray drying is typically performed at temperatures around the T_{g} of the coating agent. This may help to achieve a successful encapsulation and fabrication of a smooth and homogenous surface.

The invention also relates to a kit of parts. The kit of parts comprises a Catalyst Paste and a Base Paste.

The Catalyst Paste comprises the microcapsule(s) as described in the present text, which comprises a first component of a redox-initiator system.

The Base Paste comprises an acidic component and a second component of the redox-initiator system.

The first and second component of the redox-initiator system together form an initiator system which is suitable to initiate the curing of the curable components being present in the Catalyst Paste or the Base Paste or in the Catalyst Paste and the Base Paste.

According to one embodiment, the first component of a redox-initiator system contained in the microcapsules is a reducing agent and the second component of the redox-initiator system is an oxidizing agent.

According to another embodiment, the first component of a redox-initiator system contained in the microcapsules is an oxidizing agent and the second component of the redox-initiator system is a reducing agent. The oxidizing and reducing agents include those described above.

According to one embodiment, the kit of parts comprises two kinds of microcapsules, microcapsules containing a reducing agent and microcapsules containing an oxidizing agent.

The acidic component contained in the Base Paste is a component being suitable to interact with the gas releasing component incorporated into the polymer of the shell of the microcapsule, such that the shell is weakened (e.g. dissolved) enabling the first component of the redox-initiator system to migrate out of the pores of the porous core.

The nature and structure of the acidic component is not particularly limited unless the intended purpose cannot be achieved. Inorganic and organic acidic components can be used, as desired.

Inorganic acidic components which can be used include hydrochloric acid, sulfuric acid, phosphoric acid, mixtures thereof and its acidic salts.

Organic acidic components which can be used include monocarboxylic acids such as formic acid, acetic acid and benzoic acid and derivatives of these acids or dicarboxylic acids chosen from oxalic acid, malonic acid, succinic acid, adipic acid, pimelic acid, azelaic acid, sebacic acid, maleic acid, fumaric acid, sorbic acid, phthalic acid and terephthalic acid and derivatives of these acids or tricarboxylic acids chosen from hemimellitic acid, trimellitic acid, trimesic acid, agaric acid, citric acid, 1,2,3-propanetricarboxylic acid and derivatives of these acids or multicarboxylic acids chosen from the group consisting of pyromellitic acid and mellitic acid and derivatives of these acids or polycarboxylic acids chosen from polyacrylic acid and polymethacrylic acid and derivatives of these acids and mixtures thereof.

The acidic component can be characterized by the following features alone or in combination:
a) pKs value: equal to or below 5, equal to or below 4 or equal to or below 3.5 or equal to or below 3 or equal to or below 2;
b) comprising acidic moieties selected from sulfonic, sulfinic, phosphoric, phosphonic, phosphinic, or carboxylic moieties.

If desired, the acidic component may also comprise one or more polymerizable moieties, such as (meth)acrylate moieties. One or more polymerizable component(s) with acidic moiety(s) may be present, if desired.

The polymerizable components with acid moiety can typically be represented by the following formula

AₙBCₘ

with A being an ethylenically unsaturated group, such as a (meth)acryl moiety,

B being a spacer group, such as (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), (iii) organic group having 4 to 20 carbon atoms bonded to one another by one or more ether, thioether, ester, thioester, thiocarbonyl, amide, urethane, carbonyl and/or sulfonyl linkages, and C being an acidic group, or precursor of an acidic group such as acid anhydride, m, n being independently selected from 1, 2, 3, 4, 5 or 6, wherein the acidic group comprises one or more carboxylic acid residues, such as —COOH or —CO—O—CO—, phosphoric acid residues, such as —O—P(O)(OH)OH, phosphonic acid residues, such as C—P(O)(OH)(OH), sulfonic acid residues, such as —SO₃H or sulfinic acid residues such as —SO₂H.

Examples of polymerizable components with acid moiety include, but are not limited to glycerol phosphate mono(meth)acrylate, glycerol phosphate di(meth)acrylate, hydroxyethyl (meth)acrylate (e.g., HEMA) phosphate, bis((meth)acryloxyethyl) phosphate, (meth)acryloxypropyl phosphate, bis((meth)acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylate, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like. Derivatives of these hardenable components bearing an acid moiety that can readily react e.g. with water to form the specific examples mentioned above, like acid halides or anhydrides are also contemplated.

Also monomers, oligomers, and polymers of unsaturated carboxylic acids such as (meth)acrylic acids, aromatic (meth)acrylated acids (e.g., methacrylated trimellitic acids), and anhydrides thereof can be used.

If present, the acidic component(s) are typically present in the following amounts:
Lower amount: at least 2 or at least 3 or at least 4 wt. %;
Upper amount: utmost 50 or utmost 40 or utmost 30 wt. %;
Range: 2 to 50 or 3 to 40 or 4 to 30 wt. %;
   wt. % with respect to the weight of the composition obtained by mixing the Catalyst Paste and Base Paste of the kit of parts.

The curable component(s) which are typically contained in pastes of the kit of parts are components which can be polymerized in the presence of the redox-initiator system. According to one embodiment, the curable component(s) do not contain an acidic moiety. One or more polymerizable component(s) without acidic moiety(s) may be present, if desired. The nature and structure of those components is not particularly limited unless the intended purpose cannot be achieved.

The polymerizable component(s) without acidic moiety(s) is typically a free-radically polymerizable material, including ethylenically unsaturated monomer, monomers or oligomers or polymers.

Suitable polymerizable component(s) without acidic moiety(s) can be characterized by the following formula:

AₙBAₘ

with A being an ethylenically unsaturated group, such as a (meth)acryl moiety,
B being selected from (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), or (iii) organic group having 4 to 20 carbon atoms bonded to one another by one or more ether, thioether, ester, thioester, thiocarbonyl, amide, urethane, carbonyl and/or sulfonyl linkages,

m, n being independently selected from 0, 1, 2, 3, 4, 5 or 6 with the proviso that n+m is greater 0, that is that at least one A group is present.

Such polymerizable materials include mono-, di- or poly-acrylates and methacrylates such as methyl acrylate, methyl methacrylate, ethyl (meth)acrylate, isopropyl (meth)acrylate, n-hexyl (meth)acrylate, stearyl (meth)acrylate, allyl (meth)acrylate, glycerol di(meth)acrylate, the diurethane dimethacrylate called UDMA (mixture of isomers, e.g. Rohm Plex 6661-0) being the reaction product of 2-hydroxyethyl methacrylate (HEMA) and 2,2,4-trimethylhexamethylene diisocyanate (TMDI), glycerol tri(meth)acrylate, ethyleneglycol di(meth)acrylate, diethyleneglycol di(meth)acrylate, triethyleneglycol di(meth)acrylate, 1,3-propanediol diacrylate, 1,3-propanediol dimethacrylate, trimethylolpropane tri(meth)acrylate, 1,2,4-butanetriol tri(meth)acrylate, 1,4-cyclohexanediol di(meth)acrylate, pentaerythritol tri(meth)acrylate, pentaerythritol tetraacrylate, pentaerythritol tetramethacrylate, sorbitol hexa(meth)acrylate, bis[1-(2-(meth)acryl oxy)]-p-ethoxyphenyldimethylmethane, bis[1-(3-methacryl oxy-2-hydroxy)]-p-propoxyphenyldimethylmethane (BisGMA), bis[1-(3-acryl oxy-2-hydroxy)]-p-prop oxy-phenyldimethylmethane and trishydroxyethyl-isocyanurate trimethacrylate; the bis-acrylates and bis-methacrylates of polyethylene glycols of molecular weight 200-500, copolymerizable mixtures of acrylated monomers (see e.g. U.S. Pat. No. 4,652,274), and acrylated oligomers (see e.g. U.S. Pat. No. 4,642,126); and vinyl compounds such as styrene, diallyl phthalate, divinyl succinate, divinyl adipate and divinylphthalate; polyfunctional (meth)acrylates comprising urethane, urea or amide groups. Mixtures of two or more of these free radically polymerizable materials can be used if desired.

Further polymerizable components which may be present include di(meth)acrylates of ethoxylated bis-phenol A, for example 2,2′-bis(4-(meth)acryl-oxytetraethoxyphenyl)propanes, urethane (meth)acrylates and (meth)acrylamides. The monomers used can furthermore be esters of [alpha]-cyanoacrylic acid, crotonic acid, cinnamic acid and sorbic acid.

It is also possible to use the methacrylic esters mentioned in EP 0 235 826, such as bis[3 [4]-methacryl-oxymethyl-8(9)-tricyclo[5.2.1.0^{2,6}]decylmethyl triglycolate. Suitable are also 2,2-bis-4(3-methacryloxy-2-hydroxypropoxy)phenylpropane (Bis-GMA), 2,2-bis-4(3-methacryl oxypropoxy)phenylpropane, 7,7,9-trimethyl-4,13-di oxo-3,14-di oxa-5,12-diazahexadecane-1,16-dioxy dimethacrylate (UDMA), urethane (meth)acrylates and di(meth)acrylates of bishydroxymethyltricyclo-(5.2.1.0^{2,6})decane.

These ethylenically unsaturated monomers can be employed in the dental composition(s) either alone or in combination with the other ethylenically unsaturated monomers. In addition or besides those components, other hardenable components which can be added include oligomeric or polymeric compounds, such as polyester (meth)acrylates, polyether (meth)acrylates, polycarbonate (meth)acrylates and polyurethane (meth)acrylates. The molecular weight of these compounds is typically less than 20,000 g/mol, particularly less than 15,000 g/mol and in particular less than 10,000 g/mol.

The curable component(s) without acidic moieties are typically present in the following amounts:

Lower amount: at least 5 or at least 10 or at least 20 wt. %;

Upper amount: utmost 65 or utmost 55 or utmost 45 wt. %;

Range: 5 to 65 or 10 to 55 or 20 to 45 wt. %;

wt. % with respect to the weight of the composition obtained by mixing the Catalyst Paste and Base Paste of the kit of parts.

The Catalyst Paste and/or the Base Paste may contain further components, including filler(s), photo initiator(s) and additives including fluoride release agent(s), stabilizer(s), colorant(s).

The amounts and types of each ingredient in the composition should be adjusted to provide the desired physical and handling properties before and after polymerization.

One or more fillers may be present, if desired. The nature and structure of the filler(s) is not particularly limited unless the intended purpose cannot be achieved.

Adding a filler can be beneficial e.g. for adjusting the rheological properties like the viscosity. The content of the filler also typically influences the physical properties of the composition after hardening, like hardness or flexural strength.

The size of the filler particles should be such that a homogeneous mixture with the hardenable component forming the resin matrix can be obtained. The mean particle size of the filler may be in the range from 5 nm to 100 μm.

If desired, the measurement of the particle size of the filler particles can be done with a TEM (transmission electron microscopy) method, whereby a population is analysed to obtain an average particle diameter.

A preferred method for measuring the particle diameter can be described as follows: Samples approximately 80 nm thick are placed on 200 mesh copper grids with carbon stabilized formvar substrates (SPI Supplies—a division of Structure Probe, Inc., West Chester, Pa.). A transmission electron microscopy (TEM) is taken, using JEOL™ 200CX (JEOL, Ltd. of Akishima, Japan and sold by JEOL USA, Inc.) at 200 Kv. A population size of about 50-100 particles can be measured and an average diameter is determined.

The filler(s) typically comprise non acid-reactive fillers. A non-acid reactive filler is a filler which does not undergo an acid/base reaction with an acid.

Useful non-acid reactive fillers include fumed silica, fillers based on non-acid reactive fluoroaluminosilicate glasses, quartz, ground glasses, non water-soluble fluorides such as CaF₂, silica gels such as silicic acid, in particular pyrogenic silicic acid and granulates thereof, cristobalite, calcium silicate, zirconium silicate, zeolites, including the molecular sieves.

Suitable fumed silicas include for example, products sold under the tradename Aerosil™ series OX-50, -130, -150, and -200, Aerosil™ R8200, -R805 available from Degussa AG (Hanau, Germany), CAB-O-SIL™ M5 available from Cabot Corp (Tuscola), and HDK types e.g. HDK™-H2000, HDK™ H15, HDK™ H18, HDK™ H20 and HDK™ H30 available from Wacker.

Filler(s) which can also be used and which provide radiopacity to the dental materials described in the present text include heavy metal oxide(s) and fluoride(s). As used herein, “radiopacity” describes the ability of a hardened dental material to be distinguished from tooth structure using standard dental X-ray equipment in the conventional manner. Radiopacity in a dental material is advantageous in certain instances where X-rays are used to diagnose a dental condition. For example, a radiopaque material would allow the detection of secondary caries that may have formed in the tooth tissue surrounding a filling.

Oxides or fluorides of heavy metals having an atomic number greater than about 28 can be preferred. The heavy metal oxide or fluoride should be chosen such that undesirable colors or shading are not imparted to the hardened resin in which it is dispersed. For example, iron and cobalt would not be favoured, as they impart dark and contrasting colors to the neutral tooth color of the dental material. More preferably, the heavy metal oxide or fluoride is an oxide or fluoride of metals having an atomic number greater than 30. Suitable metal oxides are the oxides of yttrium, strontium, barium, zirconium, hafnium, niobium, tantalum, tungsten, bismuth, molybdenum, tin, zinc, lanthanide elements (i.e. elements having atomic numbers ranging from 57 to 71, inclusive), cerium and combinations thereof. Suitable metal fluorides are e.g. yttrium trifluoride and ytterbium trifluoride. Most preferably, the oxides and fluorides of heavy metals having an atomic number greater than 30, but less than 72 are optionally included in the materials of the invention. Particularly preferred radiopacifying metal oxides include lanthanum oxide, zirconium oxide, yttrium oxide, ytterbium oxide, barium oxide, strontium oxide, cerium oxide, and combinations thereof. The heavy metal oxide particles may be aggregated. If so, it is preferred that the aggregated particles are equal or less than 200 nm in average diameter.

Other suitable fillers to increase radiopacity are salts of barium and strontium especially strontium sulphate and barium sulphate.

Filler(s) which can also be used include nano-sized fillers such as nano-sized silica. Suitable nano-sized particles typically have a mean particle size in the range of 5 to 80 nm. Preferred nano-sized silicas are commercially available from Nalco Chemical Co. (Naperville, Ill.) under the product designation NALCO COLLOIDAL SILICAS (for example, preferred silica particles can be obtained from using NALCO™ products 1040, 1042, 1050, 1060, 2327 and 2329), Nissan Chemical America Company, Houston, Tex. (for example, SNOWTEX-ZL, -OL, -O, -N, -C, -20L, -40, and -50); Admatechs Co., Ltd., Japan (for example, SX009-MIE, SX009-MIF, SC1050-MJM, and SC1050-MLV); Grace GmbH & Co. KG, Worms, Germany (for example, those available under the product designation LUDOX™, e.g., P-W50, P-W30, P-X30, P-T40 and P-T40AS); Akzo Nobel Chemicals GmbH, Leverkusen, Germany (for example, those available under the product designation LEVASIL™, e.g., 50/50%, 100/45%, 200/30%, 200A/30%, 200/40%, 200A/40%, 300/30% and 500/15%), and Bayer MaterialScience AG, Leverkusen, Germany (for example, those available under the product designation DISPERCOLL™ S, e.g., 5005, 4510, 4020 and 3030).

Surface-treating the nano-sized silica particles before loading into the dental material can provide a more stable dispersion in the resin. Preferably, the surface-treatment stabilizes the nano-sized particles so that the particles will be well dispersed in the hardenable resin and results in a substantially homogeneous composition. Furthermore, it is preferred that the silica be modified over at least a portion of its surface with a surface treatment agent so that the stabilized particle can copolymerize or otherwise react with the hardenable resin during curing.

Thus, the silica particles as well as other suitable non acid-reactive fillers can be treated with a resin-compatibilizing surface treatment agent.

If present the filler(s) are typically present in the following amounts:

Lower amount: at least 1 or at least 5 or at least 10 wt. %;

Upper amount: utmost 80 or utmost 70 or utmost 60 wt. %;

Range: 1 to 80 or 5 to 70 or 10 to 60 wt. %;

wt. % with respect to the weight of the composition obtained by mixing the Catalyst Paste and Base Paste of the kit of parts. The kit of parts may also include photo-initiator(s).

The photo-initiator may be present in the Base Paste or the Catalyst Paste or in both pastes. Typically, the photo-initiator is present in the Catalyst Paste.

The nature and structure of the photo-initiator is not particularly limited unless the intended purpose cannot be achieved. Suitable photo initiator(s) for free radical polymerization are generally known to the person skilled in the art dealing with dental materials.

As photo-initiator(s), those which can polymerize the polymerizable monomer(s) by the action of visible light having a wavelength of from 350 nm to 500 nm are preferred. Suitable photo-initiator(s) often contain an alpha di-keto moiety, an anthraquinone moiety, a thioxanthone moiety or benzoin moiety.

Examples of photo-initiator(s) include camphor quinone, 1-phenyl propane-1,2-dione, benzil, diacetyl, benzyl dimethyl ketal, benzyl diethyl ketal, benzyl di(2-methoxyethyl) ketal, 4,4,′-dimethylbenzyl dimethyl ketal, anthraquinone, 1-chloroanthraquinone, 2-chloroanthraquinone, 1,2-benzanthraquinone, 1-hydroxyanthra-quinone, 1-methyl anthraquinone, 2-ethyl anthraquinone, 1-bromoanthraquinone, thioxanthone, 2-isopropyl thioxanthone, 2-nitrothioxanthone, 2-methyl thioxanthone, 2,4-dimethyl thioxanthone, 2,4-diethyl thioxanthone, 2,4-diisopropyl thioxanthone, 2-chloro-7-trifluoromethyl thioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethylaminophenyl)ketone, 4,4,′-bisdiethylaminobenzophenone.

Using acylphosphine oxides was found to be useful, as well.

Suitable acylphosphine oxides can be characterized by the following formula

(R⁹)₂—P(═O)—C(═O)—R¹⁰

wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S-, O-, or N-containing five- or six-membered heterocyclic group, or a —Z—C(═O)—P(═O)—(R⁹)₂ group, wherein Z represents a divalent hydrocarbyl group such as alkylene or phenylene having 2 to 6 carbon atoms.

Suitable systems are also described e.g. in U.S. Pat. No. 4,737,593 (Ellrich et al.), the content of which is herewith incorporated by reference.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms. In particular, 2,4,6-trimethylbenzoyl diphenyl phosphine oxide was found to be useful (Lucirin™ TPO, BASF).

Suitable bisacylphosphine oxides can also be described by the following formula:

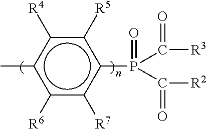

wherein n is 1 or 2, and R⁴, R⁵, R⁶ and R⁷ are H, C₁₋₄ alkyl, C₁₋₄ alkoxyl, F, Cl or Br; R² and R³, which are the same or different, stand for a cyclohexyl, cyclopentyl, phenyl, naphthyl, or biphenylyl radical, a cyclopentyl, cyclohexyl, phenyl, naphthyl, or biphenylyl radical substituted by F, Cl, Br, I, C₁₋₄ alkyl and/or C₁₋₄ alkoxyl, or an S or N-containing 5-membered or 6-membered heterocyclic ring; or R² and R³ are joined to form a ring containing from 4 to 10 carbon atoms and being optionally substituted by 1 to 6 C₁₋₄ alkyl radicals.

More specific examples include: bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide and bis-(2-chloro-1-naphthoyl)-2,5-dimethylphenylphosphine oxide.

The acylphosphine oxide bis(2,4,6-trimethylbenzoyl)phenyl phosphine oxide (previously known as IRGACURE™ 819, Ciba Specialty Chemicals) is sometimes preferred.

If present, the photo-initiator is typically present in the following amounts:

Lower amount: at least 0.1 or at least 0.2 or at least 0.3 wt. %;

Upper amount: up to 10 or up to 8 or up to 6 wt. %;

Range: 0.1 to 10 or 0.2 to 8 or 0.3 to 6 wt. %;

wt. % with respect to the weight of the composition obtained by mixing the Catalyst Paste and Base Paste of the kit of parts.

The pastes of the kit of part may contain further components including dyes, pigments, colorants, fluoride-release agents and other additive(s).

Examples of dyes or pigments, which can be used include titanium dioxide or zinc sulphide (lithopones), red iron oxide 3395, Bayferrox 920 Z Yellow, Neazopon Blue 807 (copper phthalocyanine-based dye) or Helio Fast Yellow ER. These additives may be used for individual colouring of the dental compositions.

Examples of photo-bleachable colorants which can be present include Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein and blends thereof. Further examples of photobleachable colorants can be found in U.S. Pat. No. 6,444,725.

Examples of fluoride release agents which can be present include naturally occurring or synthetic fluoride minerals. These fluoride sources can optionally be treated with surface treatment agents.

Further additives, which can be added, include stabilizers, especially free radical scavengers such as substituted and/or unsubstituted hydroxyaromatics (e.g. butylated hydroxytoluene (BHT), hydroquinone, hydroquinone monomethyl ether (MEHQ), 3,5-di-tert-butyl-4-hydroxyanisole (2,6-di-tert-butyl-4-ethoxyphenol), 2,6-di-tert-butyl-4-(dimethylamino)methylphenol or 2,5-di-tert-butyl hydroquinone, 2-(2′-hydroxy-5′-methylphenyl)-2H-benzotriazole, 2-(2′-hydroxy-5′-t-octylphenyl)-2H-benzotriazole, 2-hydroxy-4-methoxybenzophenone (UV-9), 2-(2′-hydroxy-4′,6′-di-tert-pentylphenyl)-2H-benzotriazole, 2-hydroxy-4-n-octoxybenzophenone, 2-(2′-hydroxy-5′-methacryl oxy-ethylphenyl)-2H-benzotriazole, and phenothiazine.

Further additives, which can be added, include retarder(s), (such as 1,2-diphenylethylene), plasticizers (including polyethylene glycol derivatives, polypropylene glycols, low-molecular-weight polyesters, dibutyl, dioctyl, dinonyl and diphenyl phthalate, di(isononyl adipate), tricresyl phosphate, paraffin oils, glycerol triacetate, bisphenol A diacetate, ethoxylated bisphenol A diacetate, and silicone oils), and flavorant(s).

There is no need for these adjuvants or additives to be present, so adjuvants or additives might not be present at all. However, if they are present, they are typically present in an amount which is not detrimental to the intended purpose.

If present, additive(s) is (are) typically present in the following amounts:

Lower amount: at least 0.01 wt. % or at least 0.05 wt. % or at least 0.1 wt. %;

Upper amount: utmost 15 wt. % or utmost 10 wt. % or utmost 5 wt. %;

Range: 0.01 wt. % to 15 wt. % or 0.05 wt. % to 10 wt. % or 0.1 wt. % to 5 wt. %.

The amount is given with respect to the weight of the whole composition obtained when combining the Catalyst Paste and the Base Paste.

The Catalyst Paste and the Base Paste of the kit of parts are typically stored in a packaging device during storage.

The Catalyst Paste and the Base Paste of the kit of parts described in the present text may be contained in separate sealable vessels (e.g. made out of plastic or glass).

For use, the practitioner may take adequate portions of the compositions contained from the vessels and mix the portions by hand on a mixing plate.

According to a preferred embodiment, the Catalyst Paste and the Base Paste are contained in separate compartments of a storing device.

The storing device typically comprises two compartments for storing the respective parts, each compartment being equipped with a nozzle for delivering the respective part. Once delivered in adequate portions, the parts can then be mixed by hand on a mixing plate.

According to another preferred embodiment, the storing device has an interface for receiving a static mixing tip. The mixing tip is used for mixing the respective pastes. Static mixing tips are commercially available e.g. from SulzerMixpac company. Suitable storing devices include cartridges, syringes and tubes.

The storing device typically comprises two housings or compartments having a front end with a nozzle and a rear end and at least one piston movable in the housing or compartment.

Cartridges which can be used are described e.g. in US 2007/0090079 or U.S. Pat. No. 5,918,772, the disclosure of which is incorporated by reference. Some of the cartridges which can be used are commercially available e.g. from Sulzer Mixpac AG (Switzerland). Static mixing tips which can be used are described e.g. in US 2006/0187752 or in U.S. Pat. No. 5,944,419, the disclosure of which is incorporated by reference. Mixing tips which can be used are commercially available from Sulzer Mixpac AG (Switzerland), as well.

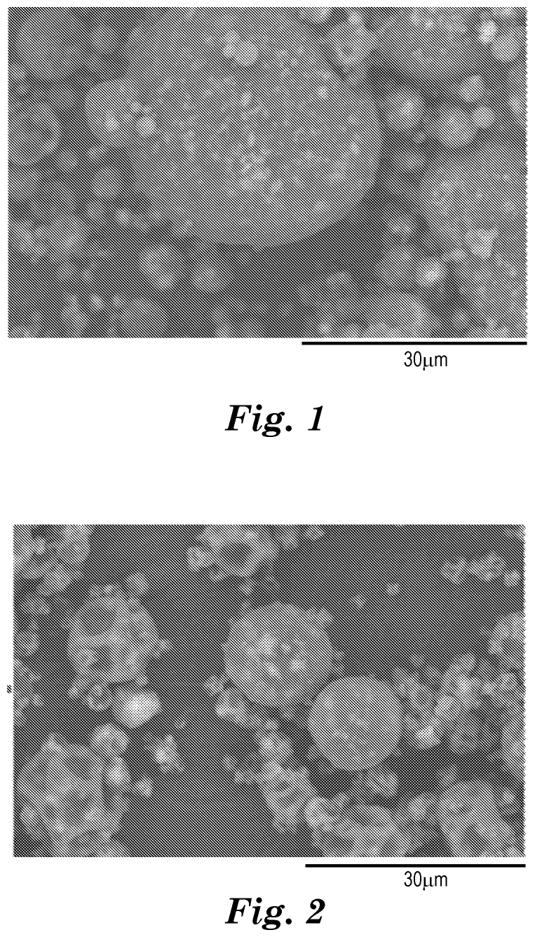

Other suitable storing devices are described e.g. in WO 2010/123800 (3M), WO 2005/016783 (3M), WO 2007/104037 (3M), WO 2009/061884 (3M), in particular the device shown in FIG. 14 of WO 2009/061884 (3M) or WO 2015/073246 (3M), in particular the device shown in FIG. 1 of WO 2015/07346. Those storing devices have the shape of a syringe. The content of these references is herewith incorporated by reference, as well.

Alternatively, but less preferred, paste/paste compositions described in the present text can be provided in two individual syringes and the individual pastes can be mixed by hand prior to use.

Thus, the invention is also directed to a device for storing the kit of parts described in the present text, the device comprising two compartments, Compartment A and Compartment B, Compartment A containing the Catalyst Paste and Compartment B containing the Base Paste, the Catalyst Paste and the Base Paste being as described in the present text, Compartment A and Compartment B both comprising a nozzle or an interface for receiving an entrance orifice of a static mixing tip.

The mixing ratio of the Base Paste and the Catalyst Base Paste is typically 3:1 to 1:3 with respect to volume, preferably 2:1 to 1:2, more preferably 1:1.

The microcapsule described in the present text is particularly useful for producing a curable composition comprising curable components and a redox-initiator system.

According to one embodiment, the curable composition is a dental or orthodontic composition.

According to one embodiment, the curable composition is a dental or orthodontic cement, adhesive or filing material.

The microcapsule described in the present text are particularly useful for producing a curable composition obtained by combining two pastes, a base paste and a catalyst paste, wherein one of the pastes contain the microcapsules described in the present text and the other paste contains an acidic component.

When mixing the two pastes, the paste containing the acidic component comes in contact with the microcapsules containing the component which produces gas upon exposure to acid in the shell. Upon contact, the shell is weakened because of gas formation. This enables the component of the redox-initiator system to be released from the porous core of the microcapsule more easily. Self-adhesive dental materials usually contain an acidic paste.

The acidity of this paste can be used as trigger to remove the shell from the microcapsule upon mixing of both pastes thereby releasing the encapsulated component or agent, in particular, the component of a redox-initiator system. The invention also relates to a process of curing a curable composition. Such a process comprises the following steps:

A Catalyst Paste comprising the microcapsules described in the present text and a Base Paste comprising an acidic or basic component is provided.

The microcapsules contain as component to be released a first component of a redox-initiator system.

Either the Catalyst Paste or the Base Paste or the Catalyst Paste and the Base Paste comprise curable components and a second component of the redox-initiator system.

The first and second component of the redox-initiator system forming an initiator system are able to initiate the curing of the curable components. The Catalyst Paste and the Base Paste are mixed.

The acidic component contained in the Base Paste dissolves or weakens the shell containing the component which produces gas upon exposure to acid, resulting in a release of the redox-initiator component contained therein.

If brought in contact with each other, the redox-initiator components initiate the curing of the curable components of the curable composition. Further suitable embodiments are described below:

According to one embodiment, the kit of parts is characterized as follows:

the Catalyst Paste comprising
the microcapsules described in the present text comprising a reducing component,
preferably a component comprising an ascorbic acid moiety,
curable non-acidic (meth)acrylate component(s),
filler(s),
   the Base Paste B comprising
acidic component(s), preferably a polymerizable component comprising an acidic
moiety,
curable (meth)acrylate component(s),
filler(s),
oxidizing component,
the reducing component and the oxidizing agent forming a redox-initiator system for curing the curable (meth)acrylate component(s).

According to another embodiment, the kit of parts is characterized as follows:

the Catalyst Paste comprising
the microcapsules described in the present text comprising an oxidizing component,
preferably a component comprising a peroxide moiety,
curable non-acidic (meth)acrylate component(s),
filler(s),
   the Base Paste B comprising
acidic component(s), preferably a polymerizable component comprising an acidic moiety,
curable (meth)acrylate component(s),
filler(s),
reducing component,
the reducing component and the oxidizing agent forming a redox-initiator system for curing the curable (meth)acrylate component(s).

The shell of the microcapsules is composed of a polymeric material which is typically soluble in an aqueous composition and which contains the component which produces gas upon exposure to acid including those described above (such as carbonate or hydrogen carbonate), preferably in a particular stage.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof. The following examples are given to illustrate the invention.

### EXAMPLES

Unless otherwise indicated, all parts and percentages are on a weight basis, all water is de-ionized water, and all molecular weights are weight average molecular weight Mw. Moreover, unless otherwise indicated all experiments were conducted at ambient conditions (23° C.; 1013 mbar).

### Materials

TABLE 1

| Compound | Abbreviation |
|---|---|
| Triethylene glycol dimethacrylate | TEGDMA |
| Urethane dimethacrylate | UDMA |
| Hydroxyethyl methacrylate | HEMA |
| 1,3-glycerol dimethacrylate phosphate | GDM-P |
| De-ionized water | H₂O |
| Glass powder G018-163 (Schott), 2.5% silanized (filler) | GP |
| Fumed silica (rheological additive) | Silica |
| Phosphite (stabilizer) | STAB |
| butylated hydroxytoluene (stabilizer) | BHT |
| Irgacure ™ 819 (photoinitiator) | IC 819 |
| BHT, Cu(Ac)₂ in GDM-P | Solution 1 |
| Hydroperoxide (oxidizing agent) | AHP |
| Ascorbic acid derivative (reducing agent) | AAD |
| Amine hydrochloride; 2 wt. % in TEGDMA (accelerator) | Amine-HCL |
| Lycoat ™ RS 780 (coating agent; Roquette Company) | Lycoat |
| Kollicoat ™ IR (coating agent; BASF) | Kollicoat |
| OX 50 filler (Evonik ™), 3.0% silanized | OX50 |
| Aerosil ® R805 (Evonik ™) | R805 |
| Aerosil ® 200 (Evonik ™) | Aerosil 200 |
| Nano calcium carbonate (particle size: 15-40 nm) | Nano-CC |
| Calcium carbonate (particle size: d98: 25 μm, d50: 5.5 μm) | CC |
| Microcapsule, filled with AAD (ascorbyl palmitate) | MCO |
| Microcapsule, filled with AAD (ascorbyl palmitate), coated | MC1 |
| with Kollicoat | |
| Microcapsule, filled with AAD (ascorbyl palmitate), coated | MC2 |
| with Kollicoat containing calcium carbonate | |
| Microcapsule, filled with AAD (ascorbyl palmitate), coated | MC3 |
| with Lycoat containing calcium carbonate | |
| Microcapsule, filled with AAD (ascorbyl palmitate) coated | MC4 |
| with Kollicoat containing nano calcium carbonate | |
| Microcapsule, filled with AAD (ascorbyl palmitate), coated | MC5 |
| with Lycoat RS 780 containing nano calcium carbonate | |
| Microcapsule, filled with AAD (ascorbyl palmitate), coated | MC6 |
| with Lycoat RS 780 | |

### Methods

### Viscosity

If desired, the viscosity can be measured using a Physica MCR 301 Rheometer (Anton Paar, Graz, Austria) with a cone/plate geometry CP25-1 under controlled shear rate at 23° C. The diameter is 25 mm, the cone angle 1°, and the separation between the cone tip and the plate 49 μm. The shear rate is ramped down logarithmically from 100 s⁻¹ to 0.001 s⁻¹.

### Scanning Electron Microscopy (SEM)

If desired, the quality of the coating process, the particle size and the shape of the microcapsules can be further analysed and determined by SEM, e.g. using the device JSM 5400 (Hitachi).

### Light-Scattering

If desired, the particle size distribution can be determined by light-scattering, e.g. using the device Horiba (Horiba, JP). The thickness of the shell can further be calculated based on its shape and dimension using e.g. geometrical equations and relations.

### Mechanical Stability

If desired, the mechanical stability of the microcapsules can be determined as follows:

The microcapsules to be analysed are filled with Sudan blue II (a dye having a blue color). The filled microcapsules are then coated with a coating agent. Pastes are then prepared using e.g. the following composition: 18 wt. % TEGDMA, 20 wt. % UDMA, 52.02 wt. % glass filler, 8.0 wt. % fumed silica, 0.1 wt. % IC 819, 0.46 wt. % coated filled microcapsules.

The composition is mixed by using a commercially available speed mixer (e.g. SpeedMixer™ DAC 150 SP; Hauschild, Germany) applying the following conditions: 3×90 s, 2500 RPM and 3×20 s 3500 RPM with cooling to room temperature after each mixing step.

From this composition light-cured discs are prepared: 700 mg of the paste is filled into a cylindrical mold (15 mm diameter; 1.5 mm height) which is placed between glass slides covered with transparency films. This sandwich-like structure is irradiated from both sides for 20 s (without light guide) using an Elipar™ S10 light curing device (3M Oral Care).

Then, the specimen is removed from the mold and put into a Visio™ Beta Vario light oven with vacuum (3M Oral Care) for 7 min to fully light cure the sample.

Then, the L*a*b* color coordinates are determined. If the b* value is positive, the blue color Sudan Blue II obviously did not release from the microcapsules during the paste and disc preparation. This is an indication that the tested microcapsules are mechanically stable. If the b* value is negative, the blue color Sudan Blue II obviously did release from the microcapsules during the paste and disc preparation. This is an indication that the tested microcapsules are mechanically not stable.

### Determination of Working & Setting Time

A Physica MCR 301 Rheometer (Anton Paar, Graz, Austria) with a plate/plate geometry of 8 mm diameter at a temperature of 28° C. was used. 100 mg of Paste Ax was hand mixed with 100 mg of Paste PB1 (mixing ratio 1.0:1.0 w/w) for 20 s on a mixing pad with the help of a spatula. Then, the mixture was applied between the plates and the gap was set to 0.75 mm. Frequency was 1.25 Hz, oscillation with 1.75% deflection.

The working time (Ta) is defined as the time between start of mixing and time of reaching intersection point of G′ and G″. The setting time (Tf) is defined as the time between start of mixing and the time for the mixed pastes to reach a shear stress of 100,000 Pa.

### Preparation of Microcapsules

The monomers SR 339 (100 grams) and SR 6030P (100 grams) obtained from Sartomer and sulfo-ethyl-methacrylate (10 grams) were mixed with Acclaim™ Polyol PPG 4200 (86 grams) from Covestro and IRGACURE™ 819 (600 milligrams) from BASF. The mixture was stirred vigorously for 20 minutes. This mixture was then added to 1200 grams of glycerol previously mixed with 36 g of the surfactant APG 325 obtained from Cognis Corporation. The mixture was shear-mixed for 10 min with a high shear mixer.

The mixture was then spread thin between to sheets of polyethylene terephthalate (PET) and cured with ultraviolet light for 15 minutes with a 6 Watts, long-wavelength UVA lamp (obtained from UVP, LLC of Upland, Calif., USA) situated at about 10 centimeters from the surface of the curing material.

The cured mixture was then dispersed in four bottles in isopropyl alcohol (300 mL) and centrifuged at 3000 rpm. The supernatant was removed and the resulting particles were re-suspended in four bottles with 500 mL of isopropyl alcohol for a second rinse followed by centrifugation. After this, the particles were suspended in 4 bottles a 300 mL isopropyl alcohol and shaken for 2 min and centrifuged again. This extracted the PPG and left pores in the particles.

Filling of Microcapsules with Component to be Released

The porous polymer particles can be filled with a component to be released, for example an initiator or crosslinker. The filling is realized by adding the porous polymer particles carefully under mixing to a liquid active or a dilution of an active. The solvent should be removed afterwards by drying.

To prepare the MC0 sample 6 g ascorbyl palmitate were mixed with 22 g THF. This liquid mixture was placed in a glass dish. 20 g of the porous particles were added with continuous stirring by hand.

Coating of Filled Microcapsules with Calcium Carbonate Containing Polymer

The filled porous polymer particles were coated with a shell via spray-drying to form microcapsules. For a lab scale a spray drier from Buchi, Type B 290 with an inlet temperature range of 55° C. to 110° C. was used.

For example, MC2 was prepared by mixing 5 g MC0 with 100 g of a 20 wt. % solution of Kollicoat containing 5 g calcium carbonate. Optionally, a surfactant such as APG™ 325 (BASF) can be added.

Two different calcium carbonate grades were used: nano calcium carbonate (Nano-CC) and calcium carbonate (CC).

The experiments were performed with seven different capsule types:
1. MC0: microcapsules filled with AAP.
2. MC1: MC0 coated with Kollicoat.
3. MC2: MC0 coated with Kollicoat containing CC.
4. MC3: MC0 coated with Lycoat containing CC.
5. MC4: MC0 coated with Kollicoat containing Nano-CC.
A SEM picture of the obtained microcapsules is shown in FIG. 1.
6. MC5: MC0 coated with Lycoat containing Nano-CC.
A SEM picture of the obtained microcapsules is shown in FIG. 2.
7. MC6: MC0 coated with Lycoat.

### Compositions/Pastes

### Paste PAx

Pastes PAx were prepared by weighing in the respective components shown in Table 5.

The pastes were obtained by using a commercially available SpeedMixer™ DAC 150 SP (Hauschild, Germany) by application of 3×90 s, 2500 RPM and 3×20 s 3500 RPM (cooling to room temperature after each mixing step).

The capsule types contain different amounts of ascorbyl palmitate. Therefore, weight percentages in Table 2 had to be adjusted accordingly to make sure that microcapsules with the same amount of ascorbyl palmitate were compared (see Table 5).

TABLE 2

| | PA1 | PA2 | PA3 | PA4 | PA5 | PA6 | PA7 |
|---|---|---|---|---|---|---|---|
| Component | Amount [wt. %] | | | | | | |
| TEGDMA | 18.0 | 18.0 | 18.0 | 18.0 | 18.0 | 18.0 | 18.0 |
| UDMA | 20.0 | 20.0 | 20.0 | 20.0 | 20.0 | 20.0 | 20.0 |
| GP | 50.4 | 46.6 | 46.6 | 46.6 | 46.6 | 47.3 | 47.3 |
| R805 | 8.0 | 8.0 | 8.0 | 8.0 | 8.0 | 8.0 | 8.0 |
| MC0 | 1.0 | — | — | — | — | — | — |
| MC1 | — | — | — | — | — | — | 4.1 |
| MC2 | — | 4.8 | — | — | — | — | — |
| MC3 | — | — | 4.8 | — | — | — | — |
| MC4 | — | — | — | 4.8 | — | — | — |
| MC5 | | | | | 4.8 | — | — |
| MC6 | | | | | | 4.1 | — |
| STAB | 0.5 | 0.5 | 0.5 | 0.5 | 0.5 | 0.5 | 0.5 |
| Amine-HCl | 2.0 | 2.0 | 2.0 | 2.0 | 2.0 | 2.0 | 2.0 |
| IC 819 | 0.1 | 0.1 | 0.1 | 0.1 | 0.1 | 0.1 | 0.1 |

Paste PB1

Paste PB1 was prepared by weighing in the respective components shown in Table 6.

The paste was obtained by using a commercially available SpeedMixer™ DAC 150 SP (Hauschild, Germany) by application of 3×90 s, 2500 RPM and 2×60 s 3500 RPM (cooling to room temperature after each mixing step).

TABLE 3

| | PB1 |
|---|---|
| | Amount |
| Component | [wt. %] |
| HEMA | 20.0 |
| GDM-P | 15.0 |
| H2O | 10.0 |
| OX50 | 45.2 |
| Aerosil 200 | 1.5 |
| Solution 1 | 6.0 |
| AHP | 2.3 |

### Curable Compositions

Curable compositions according to Examples 1 to 7 shown in Table 7 were prepared by mixing Pastes PAx with Paste PB1 (mixing ratio 1.0:1.0 w/w). The mixing was done by hand using a mixing pad.

TABLE 4

| Example | Paste | Paste |
|---|---|---|
| 1 | PA1 | PB1 |
| 2 | PA2 | PB1 |
| 3 | PA3 | PB1 |
| 4 | PA4 | PB1 |
| 5 | PA5 | PB1 |
| 6 | PA6 | PB1 |
| 7 | PA7 | PB1 |

Working and setting time of the obtained composition were determined. The results are given in Table 5.

TABLE 5

| | | | | | Setting |
|---|---|---|---|---|---|
| | AAP | | Calcium | Working | Time |
| Example | [wt. %] | Polymer Shell | Carbonate | Time [min] | [min] |
| 1 | 0.12 | — | — | 2.7 | 3.7 |
| 2 | 0.12 | Kollicoat | CC | 5.5 | 8.0 |
| 4 | 0.12 | Kollicoat | Nano-CC | 6.5 | 10.0 |
| 7 | 0.12 | Kollicoat | — | >30.0 | >30.0 |
| 3 | 0.12 | Lycoat | CC | 7.6 | 11.2 |
| 5 | 0.12 | Lycoat | Nano-CC | 6.3 | 10.4 |
| 6 | 0.12 | Lycoat | — | 10.5 | 17.1 |

The microcapsules filled with an active agent to be released provided a working and setting time in the desired range (Example 1).

If the microcapsules were coated with either Kollicoat™ IR or Lycoat™ RS 780 working and setting time was not completely adequate for dental applications (Examples 6 and 7).

However, the incorporation of calcium carbonate into the polymer shell shortened the working and setting time, enabling a fast release of the active agent out of the microcapsule (Examples 2, 3, 4 and 5).

The release of the active agent could even be improved, if a nano-sized calcium carbonate was used (Examples 4 and 5).

1. A microcapsule comprising
a hollow or porous core, the hollow or porous core being composed of a crosslinked polymeric material and comprising a component to be released,
a shell, the shell being composed of a polymeric material and comprising a component, which produces gas upon exposure to acid,
the crosslinked polymeric material of the hollow or porous core comprising a (meth)acrylate.

2. The microcapsule according to claim 1, the component which produces gas upon exposure to an acid being characterized by the following features alone or in combination
having a molecular weight in the range of 68 to 200 g/mol;
having the shape of particles with a particle size in the range of 5 nm to 25 μm.

3. The microcapsule according to claim 1, the component which produces gas upon exposure to acid comprising a moiety selected from carbonate, hydro carbonate or mixtures thereof.

4. The microcapsule according to claim 1, the ratio of polymeric material of the shell to component which produces gas upon exposure to acid being in a range of 2:1 to 20:1 with respect to weight.

5. The microcapsule according to claim 1, the porous or hollow core being characterized by the following features alone or in combination
having an essentially spherical shape;
having a diameter in the range of 1 to 200 μm;
the pore size of the porous core material being in the range of 10 to 200 nm;
being mechanically stable.

6. The microcapsule according to claim 1, the shell being characterized by the following features alone or in combination:
thickness: 0.1 to 5 μm;
covering more than 85% of the surface of the porous or hollow core;
insoluble in organic monomers;
soluble in an aqueous composition with a water content of 10% or more.

7. The microcapsule according to claim 1, the polymeric material of the shell comprising components selected from hydroxypropyl modified pea-starch, graft co-polymer(s) of vinyl alcohol and ethylene glycol, co-polymer(s) of methyl (meth)acrylate and diethylaminoethyl (meth)acrylate, co-polymer(s) of methyl (meth)acrylate, butyl methacrylate and dimethylaminoethyl (meth)acrylate, cellulose acetate phthalate, sodium carboxymethyl cellulose, hydroxy propyl methyl cellulose phthalate, polyvinyl acetate phthalate, co-polymer(s) of methacrylic acid and methyl (meth)acrylate, co-polymer(s) of methacrylic acid and alkyl (meth)acrylate, co-polymer(s) of methacrylic acid, methyl (meth)acrylate and methyl acrylate, and mixture thereof.

8. The microcapsule according to claim 1, the component to be released being a component of a redox-initiator system, preferably a component being selected from a reducing, oxidizing agent or transition metal component.

9. The microcapsule according to claim 1, the polymeric material of the shell comprising components selected from hydroxypropyl modified pea-starch, graft co-polymer(s) of vinyl alcohol and ethylene glycol, co-polymer(s) of methyl (meth)acrylate and diethylaminoethyl (meth)acrylate, co-polymer(s) of methyl (meth)acrylate, butyl methacrylate and dimethylaminoethyl (meth)acrylate, cellulose acetate phthalate, sodium carboxymethyl cellulose, hydroxy propyl methyl cellulose phthalate, polyvinyl acetate phthalate, co-polymer(s) of methacrylic acid and methyl (meth)acrylate, co-polymer(s) of methacrylic acid and alkyl (meth)acrylate, co-polymer(s) of methacrylic acid, methyl (meth)acrylate and methyl acrylate, and mixture thereof,
the component which produces gas upon exposure to acid comprising a moiety selected from carbonate, hydro carbonate or mixtures thereof,
the component to be released being a component of a redox-initiator system, preferably a component being selected from a reducing, oxidizing agent or transition metal component.

10. A process of producing the microcapsule according to claim 1, the process comprising the steps of:
providing a particle with a hollow or porous core being composed of a crosslinked polymeric material, the crosslinked polymeric material of the hollow or porous core comprising a (meth)acrylate,
providing a component to be released,
having the particle absorb the component to be released,
coating the particle containing the absorbed component with a polymeric coating material comprising a component, which releases gas upon exposure to acid, the coating step being preferably done by spray-drying, optionally in combination with a surfactant.

11. Use of the microcapsules described in claim 1 for producing a curable composition comprising curable components and a redox-initiator system, in particular, a dental or orthodontic composition, such as a dental or orthodontic cement, adhesive or filling material, wherein the microcapsules contain one component of the redox-initiator system.

12. A process of curing a curable composition, the process comprising the steps:
providing a Catalyst Paste comprising the microcapsules according to claim 1 and a Base Paste comprising an acidic component,
the microcapsules containing a first component of a redox-initiator system to be released,
the Base Paste comprising a second component of a redox-initiator system,
either the Catalyst Paste or the Base Paste or the Catalyst Paste and the Base Paste further comprising curable components,
mixing the Catalyst Paste and the Base Paste.

13. A kit of parts comprising a Catalyst Paste and a Base Paste,
the Catalyst Paste comprising
the microcapsule as described in claim 1 comprising a first component of a redox-initiator system,
the Base Paste comprising
an acidic component,
a second component of the redox-initiator system.

14. The kit of parts according to claim 13, the Base Paste containing an acidic component being characterized by the following features alone or in combination:
pKs value: below 5;
comprising an acidic moiety selected from a sulfonic, sulfinic, phosphoric, phosphonic, phosphinic, or carboxylic moiety;
comprising one or more polymerizable moieties.

15. The kit of parts according to claim 13 being characterized as follows:
the Catalyst Paste comprising
the microcapsules as described in claim 1 comprising a reducing agent,
curable non-acidic (meth)acrylate component(s),
filler(s),
optionally photo initiator(s),
the Base Paste comprising
acidic component(s),
curable (meth)acrylate component(s),
filler(s),
oxidizing agent,
optionally transition metal component(s),
the reducing component and the oxidizing agent forming a redox-initiator system for curing the curable (meth)acrylate component(s).
